SEQUENCE LISTING (12 sequences)

SEQ ID NO: 1 - DNA, 4461 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattcttatattca

SEQ ID NO: 2 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 3 - DNA, 1061 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 4 - RNA, 19 nt, Artificial Sequence
  siRNA N-Ras sense
gucauuugcggauauuaac

SEQ ID NO: 5 - RNA, 19 nt, Artificial Sequence
  siRNA N-Ras antisense
guuaauauccgcaaaugac

SEQ ID NO: 6 - DNA, 47 bp, Artificial Sequence
  DNA Sequence encoding shRNA N-Ras
gtcatttgcggatattaacttcaagagagttaatatccgcaaatgac

SEQ ID NO: 7 - RNA, 19 nt, Artificial Sequence
  siRNA K-Ras sense
ggcauacuaguacaagugg

SEQ ID NO: 8 - RNA, 19 nt, Artificial Sequence
  siRNA K-Ras antisense
ccacuuguacuaguaugcc

SEQ ID NO: 9 - DNA, 47 bp, Artificial Sequence
  DNA Sequence encoding shRNA K-Ras
ggcatactagtacaagtggttcaagagaccacttgtactagtatgcc

SEQ ID NO: 10 - RNA, 19 nt, Artificial Sequence
  siRNA H-Ras sense
uauaagcuggugguggugg

SEQ ID NO: 11 - RNA, 19 nt, Artificial Sequence
  siRNA H-Ras antisense
ccaccaccaccagcuuaua

SEQ ID NO: 12 - DNA, 47 bp, Artificial Sequence
  DNA Sequence encoding shRNA H-Ras
ggagcgatgacggaatatatttgtgtagtatattccgtcatcgctcc

(19) United States
(12) Patent Application Publication
(10) Pub. No.: US 2011/0307054 A1
(43) Pub. Date: Dec. 15, 2011

(54) IMPLANT COATING WITH NUCLEIC ACIDS

(76) Inventors: Alexander Borck, Aurachtal (DE); Matthias Gratz, Erlangen (DE)

(21) Appl. No.: 12/959,684

(22) Filed: Dec. 3, 2010

Related U.S. Application Data
(60) Provisional application No. 61/266,520, filed on Dec. 4, 2009.

(51) Int. Cl. A61F 2/82; A61K 31/7088; A61P 43/00; C07H 21/04; A61K 9/14; A61K 9/00; C07H 21/02

(52) U.S. Cl. 623/1.46; 424/400; 514/44 A; 536/24.5; 424/499; 424/484

(57) ABSTRACT

One example embodiment of the present invention relates to an implant with a coating or a cavity filling containing a nucleic acid, which i) can inhibit the expression of at least one representative of the Ras gene family by means of RNA interference; or ii) encodes for a nucleic acid that can inhibit the expression of at least one representative of the Ras gene family by means of RNA interference.

### CROSS REFERENCE

The present application claims priority on U.S. Provisional Application No. 61/266,520, filed on Dec. 4, 2009, which is incorporated herein by reference.

### FIELD

A field of the invention is medical implants. Another field is stents.

### BACKGROUND OF THE INVENTION

Implants are used in many different forms in modern medical engineering. For example, they are used to support vessels, hollow organs and duct systems (endovascular implants, e.g., stents) to attach and temporarily fix tissue implants and tissue transplants, but also for orthopedic purposes, for example, as nails, plates or screws. Often only a temporary support function or holding function until the end of the healing process or the stabilization of the tissue is necessary or desirable. In order to avoid complications that result from the implants remaining permanently in the body, the implants must either be surgically removed again or they are composed of a material that is gradually degraded in the body, that is, is biocorrodible. The number of biocorrodible materials on the basis of polymers or alloys has grown steadily. Thus, among other things, biocorrodible metal alloys of the elements magnesium, iron and tungsten are known. One form of an implant that is used particularly frequently is the stent.

The implantation of stents has become established as one of the most effective therapeutic measures in the treatment of vascular disease. The purpose of stents is to take over a support function in hollow organs of a patient. Stents of a conventional design for this purpose have a filigree support structure of metallic struts, which structure is initially in a compressed form for introduction into the body and is dilated at the application site. One of the main areas of application of stents of this type is permanently or temporarily expanding and keeping open vascular constrictions, in particular constrictions (stenoses) of the coronary vessels. In addition, for example, aneurysm stents are known, which are used to support damaged vascular walls.

Stents have a circumferential wall of sufficient supporting force to hold open the constricted vessel to the desired extent, and a tubular base body through which the blood flow continues to flow unimpeded. The circumferential wall can be formed by a latticed support structure that makes it possible to insert the stent in a compressed state with a small external diameter up to the constricted point of the respective vessel to be treated and to expand it there, for example, with the aid of a balloon catheter, to the extent that the vessel has the desired enlarged internal diameter. The process of positioning and expanding the stent during the procedure and the final position of the stent in the tissue after the end of the procedure must be monitored by the cardiologist. This can be carried out by imaging methods, such as, e.g., by means of x-ray examinations.

The stent has a base body of an implant material. An implant material is a nonviable material that is used for an application in medicine and interacts with biological systems. The basic prerequisites for the use of a material as an implant material, which comes into contact with the physical environment when used for its intended purpose, is the physical compatibility thereof (biocompatibility). Biocompatibility means the ability of a material to induce a suitable tissue reaction in a specific application. This entails an adaptation of the chemical, physical, biological and morphological surface properties of an implant to the recipient tissue with the objective of a clinically desired interaction. The biocompatibility of the implant material furthermore depends on the chronological course of the reaction of the biosystem into which the implant is placed. For example, irritations and inflammations, which can lead to tissue changes, occur in the relatively short term. Biological systems accordingly react in different ways depending on the properties of the implant material. According to the reaction of the biosystem, the implant materials can be subdivided into bioactive, bioinert and degradable/reabsorbable materials.

A biological reaction to polymeric, ceramic or metallic implant materials depends on the concentration, exposure time and type of administration. Often the presence of an implant material leads to inflammatory reactions, the triggers of which can be mechanical irritations, chemical substances or metabolic products. The inflammation process as a rule is accompanied by the immigration of neutrophilic granulocytes and monocytes through the vessel walls, the immigration of lymphocyte effector cells with the formation of specific antibodies against the inflammatory irritation, the activation of the complement system with the release of complement factors, which act as mediators, and ultimately the activation of blood clotting. An immunological reaction is usually closely connected to the inflammatory reaction and can lead to sensitization and the development of allergies. Known metallic allergens comprise, for example, nickel, chromium and cobalt, which are also used as alloying constituents in many surgical implants. A major problem in stent implantation in blood vessels is the in-stent restenosis due to excessive neointimal growth which is caused by a strong proliferation of the arterial smooth muscle cells and a chronic inflammatory reaction.

### SUMMARY OF THE INVENTION

One object of an embodiment of the present invention is to prevent or avoid at least one of the disadvantages of the prior art.

This and other objects are attained by providing an implant with a coating or a cavity filling containing at least one nucleic acid which
i) Can inhibit the expression of at least one representative of the Ras gene family by means of RNA interference; or
ii) Encodes for a nucleic acid that can inhibit the expression of at least one member of the Ras gene family by means of RNA interference.

The solution according to some invention embodiments provides that the expression and function of at least one representative of the Ras family, but preferably several representatives of the Ras family in parallel is inhibited in a targeted manner by means of RNA interference (RNAi) technology, in the cells surrounding the implant and thus an undesirable neointimal formation can be reduced or avoided completely. If the implant is a stent, a restenosis can be prevented or the formation of a restenosis can be retarded through the coating or cavity filling according to the invention. To this end, an implant embodiment has a coating and/or a cavity filling, which contains nucleic acids that are either able themselves to inhibit the expression of at least one Ras representative in a target cell via an RNAi effect, or encode for a nucleic acid of this type, so that after uptake by a target cell the corresponding nucleic acid can be transcribed and the transcript can develop its inhibiting effect via an RNAi effect. One advantage of the RNAi system presented is that inhibitors of different Ras representatives can thus be effectively combined in a coating, since the inhibitors are very similar in their physiochemical properties and due to the high specificity of the nucleic acids with RNAi effect desired Ras representatives can be inhibited in a targeted manner without having to anticipate any appreciable side effects on the expression or function of other genes and/or proteins. After the application of the implant according to the invention has taken place, the nucleic acid can emerge from the coating or the cavity filling, be taken up by the surrounding cells and develop the desired RNAi effect in these target cells.

The implant is preferably composed entirely or in part of a biocorrodible metallic material. This biocorrodible metallic material is preferably a magnesium alloy.

The implant according to some invention embodiments is preferably a stent.

The implant preferably has a metallic base body. In particular, some metallic base body embodiments are composed of magnesium, a biocorrodible magnesium alloy, pure iron, a biocorrodible iron alloy, a biocorrodible tungsten alloy, a biocorrodible zinc alloy or a biocorrodible molybdenum alloy.

Another example embodiment of the invention is a nucleic acid containing a sequence with one or more of the SEQ ID NOs: 4, 5, 6, 7, 8, 9, 10, 11 and 12 or of a sequence that is ≧80%, in some other embodiments ≧90%, in still further embodiments ≧95%, in still further embodiments ≧98%, in still further embodiments ≧99%, and in still further embodiments ≧99.5%, identical to SEQ ID NOs: 4, 5, 6, 7, 8, 9, 10, 11 and/or 12 or the reverse complement thereof for preventing and/or inhibiting restenosis.

### DETAILED DESCRIPTION OF THE INVENTION

Embodiments of the invention include implants, with one example being a stent. Some invention embodiments include nucleic acid sequences, with SEQ ID NOs: 1-12 provided herewith and forming a part of the present disclosure.

Implant materials for stents comprise polymers, metallic materials and ceramic materials (for example, as a coating). Biocompatible metals and metal alloys for permanent implants contain, for example, stainless steels (for example, 316L). Cobalt-based alloys (for example, CoCrMo cast alloys, CoCrMo forge alloys, CoCrWNi forge alloys and CoCrNiMo forge alloys), pure titanium and titanium alloys (e.g., CP titanium, TiA16V4 OR TiA16Nb7) and gold alloys. In the field of biocorrodible stents, the use of magnesium or pure iron as well as biocorrodible base alloys of the elements magnesium, iron, zinc, molybdenum and tungsten are proposed.

Embodiments of the invention utilize knowledge that a higher level of biocompatibility and thus an improvement in the restenosis rate can be achieved when implant materials are provided with coatings of particularly tissue-tolerant materials. These materials are usually of an organic or synthetic-polymer nature and partially of natural origin. Further strategies for avoiding restenosis are concentrated on inhibiting proliferation through medication, e.g., treatment with antitumor agents. The active substances can be provided on the surface of the implant in the form of a coating, for example. A suppression of the Ras-mediated signal transduction is a very promising way of inhibiting cell proliferation. One problem of the prior art is that there is a high level of redundancy in the Ras system. The cell can express several different proteins of the Ras family, which at least in part can compensate for the inhibition of a specific representative of the Ras family. An effective influence on the neointimal proliferation is very difficult due to this redundancy. Embodiments of the present invention address this.

For the purposes of the invention, alloys and elements are described as biocorrodible in which a degradation/conversion occurs in the physiological environment so that the part of the implant composed of the material is no longer present entirely or at least chiefly.

Magnesium alloy, iron alloy, zinc alloy, molybdenum alloy or tungsten alloy in this case mean a metallic structure the main component of which is magnesium, iron, zinc, molybdenum or tungsten. The main component is the alloying constituent with the highest weight percentage in the alloy. A percentage of the main component is preferably more than 50% by weight, in particular more than 70% by weight. The alloy should be selected in its composition such that it is biocorrodible. Synthetic plasma is used as a test medium for testing the corrosion behavior of a possible alloy, as is stipulated for biocorrosion tests according to EN ISO 10993-15:2000 (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the alloy to be tested is stored in a closed sample container with a defined quantity of the test medium at 37° C. At intervals—coordinated with the anticipated corrosion behavior—of a few hours to several months, the samples are removed and tested for traces of corrosion in the known manner. The synthetic plasma according to EN ISO 10993-15:2000 corresponds to a hematoid medium and thus represents a way of readjusting a physiological environment as defined by the invention in a reproducible manner.

According to some invention embodiments, an implant has a coating or a cavity filling that contains a nucleic acid. A coating for the purposes of the invention is an application at least in some sections of the components of the coating onto the base body of the implant. In some embodiments the entire surface of the base body of the implant is covered by the coating. An example layer thickness is in the range of 1 nm to 100 μm, and another 300 nm to 15 μm. The coating can be applied directly onto the implant surface. The processing can be carried out according to standard methods for the coating. Single-layer as well as multilayered systems (for example, so-called basecoat layers, drug coat layers or top coat layers) can be produced. The coating can be applied directly onto the base body of the implant or further layers are provided there between.

Alternatively, the nucleic acid can be a constituent of a cavity filling. The cavity is generally located on the surface of the implant. In the case of stents with a biodegradable base body, the cavity can also be arranged in the interior of the base body so that the release of the nucleic acid does not occur until after it is exposed. As an example, some cavities of invention embodiments are initially enclosed but become exposed over time as a portion of the base body (and/or a coating) decay. The cavity can optionally be open or covered by a further coating.

Methods for coating implants and for applying cavity fillings to implants are known to one skilled in the art. Some examples include vapor deposition, liquid spraying, immersion, and the like.

In addition to the use of nucleic acids according to the invention, the coating or cavity filling can also contain further constituents, in particular a polymer matrix in which the nucleic acid is embedded. In some embodiments it is embedded in a finely dispersed form. In other words, some implant embodiments have a coating or cavity filling that comprises or is composed of a polymeric, preferably organic matrix with embedded nucleic acids. The matrix can in particular contain further pharmaceutical active substances, x-ray markers or magnetic resonance markers.

In particular, the coating or cavity filling of the implant according to some invention embodiments can contain the nucleic acid in the form of nucleic acid-loaded nanoparticles. Nanoparticle is used here to describe particles with an average diameter of <1 μm. Preferably, nanoparticles are used that are loaded with siRNA molecules. For the production of nanoparticles loaded with nucleic acids, polycations are suitable such as poly-1-lysine (PLL), chitosan, poly(diallyldimethyl ammonium chloride) (polyDADMAC), dimethylaminoethyl acrylate, dimethylaminoethyl methacrylate, dimethylammonium propylmethacrylamide, dimethylaminomethyl acrylamide, acryloxy ethyltrimethylammonium chloride, methacryloxyethyl trimethylammonium chloride, methacrylamido propyl trimethyl ammonium chloride, 3-methacryloxy-2-hydroxy-propyltrimethylammonium chloride, (3-acrylamido-3-methyl) butyl trimethyl ammonium chloride, polyethylenamine, polyethylenimine, cationic dendritic polymers, diethylaminoethyl dextran, polyamidoamine, carboxymethyl cellulose, polyesters of lactic acid and copolymers thereof with glycolic acid such as PGLA. To reduce cytotoxic properties of the polycations, these can be rendered more compatible by the use of block copolymers with polyethylene glycol. To this end, the polycation, e.g., PLL as a PLL-hydrobromide, can be converted with, for example, an N-hydroxysuccinimidyl ester of a methoxy-terminated polyethylene glycol.

Preferably, the nucleic acid-loaded nanoparticles can be produced as follows. The nucleic acid, preferably in the form of RNA, particularly preferably in the form of double-stranded RNA, very particularly preferably in the form of siRNA or shRNA, is added to a polycation solution, preferably while being stirred. The concentration of polycations is thereby determined according to the quantity of nucleic acid used. A suitable ratio can be easily determined by one skilled in the art using routine tests. Suitable ratios of possible charges (e.g., amino groups) of the polycation to the phosphate groups of the nucleic acid (N+/P−) are in the range of 2:1 to 10:1, a ratio of 3:1 is preferred. In the case of an excess of positive charges, in particular with a ratio of 3:1, nanoparticles of <200 nm are obtained, which have particularly favorable transfection properties.

The implant according to some invention embodiments has a coating or a cavity filling containing at least one nucleic acid. “Nucleic acid” thereby means individual nucleotides and polymers of nucleotides. The term “nucleic acid” covers DNA as well as RNA and DNA/RNA hybrids. If a nucleic acid has a certain sequence, this sequence is given in 5′-3′ direction. According to the invention, a nucleic acid can have naturally occurring nucleotides such as A, G, T, C U, but a nucleic acid can also have one or more modified and or synthetic nucleotides or nucleotide analogs. In the attached sequence listing, the nucleic acid sequences are given either for the case that the nucleic acid sequence is an RNA molecule or that it is a DNA molecule. Of course, the given sequences also include the corresponding sequence in the respectively other nucleic acid class. The sequences with the SEQ ID NOs: 4 through 12 according to the invention include RNA, DNA as well as RNA/DNA hybrids with the given sequence. The nucleotide U (Uracil) of an RNA sequence is thereby replaced by the nucleotide T (Thymine) in order to represent a DNA sequence and vice versa.

The at least one nucleic acid of the coating or cavity filling according to some invention embodiments can either itself inhibit the expression of at least one representative of the Ras gene family by means of RNA interference (RNAi) or encode for a nucleic acid that can inhibit the expression of at least one representative of the Ras gene family by means of RNAi.

The Ras gene family covers a family of genes that encode for proteins with GTPase function, which play a crucial role in a number of cellular signal transductions, in particular the mediation of cell proliferation. Representatives of the Ras gene family are characterized in that they encode for proteins which have a so-called G domain and a so-called C terminal membrane-targeting region (CAAX-COOH, also known as CAAX box), which can be lipid-modified. A current summary of the Ras family can be found, for example, in Wennerberg K, Rossmann K L, Der C J (2005) The Ras superfamily at a glance, J Cell Sci. 118: 843-6. In particular, the Ras gene family comprises the following representatives: DIRAS1; DIRAS2; DIRAS3; ERAS; GEM; H-RAS; K-RAS; MRAS; NKIRAS1; NKIRAS2; N-RAS; RALA; RALB; RAPIA; RAPIB; RAP2A; RAP2B′; RAP2C; RASD1; RASD2; RASL10A; RASL10B; RASL11A; RASL11B; RASL12; REM1; REM2; RERG; RERGL; RRAD; RRAS; RRAS2. Preferred representatives of the Ras gene family are N-Ras with the SEQ ID NO: 1, K-Ras with the SEQ ID NO: 2 and/or H-Ras with the SEQ ID NO: 3, wherein respectively the cDNA sequences of the respective representatives of the Ras gene family are given here, Preferably, the implant according to the invention has a coating or a cavity filling that contains a nucleic acid, which can inhibit the expression of at least one of the genes N-Ras with the SEQ ID NO: 1, K-Ras with the SEQ ID NO: 2 and/or H-Ras with the SEQ ID NO: 3 or a gene having cDNA that is ≧80%, preferably ≧90%, particularly preferably ≧95%, particularly preferably ≧98%, particularly preferably ≧99%, particularly preferably ≧99.5% identical to one of the sequences with the SEQ ID NOs: 1, 2 and/or 3.

According to some embodiments of the invention, the at least one nucleic acid of the coating or the cavity filling itself can inhibit the expression of at least one representative of the Ras gene family by means of RNAi. The gene expression can be inhibited post-transcriptionally through the presence of double-strand RNA fragments, which have regions that are homologous to the sequence of the mRNA of the gene to be suppressed. This process is referred to as RNA interference or RNAi. An overview of the current use of RNAi technology can be found in Wadhwa et al. (2004), Know-how of RNA interference and its application in research and therapy, Mutat Res. 567(1): 71-84.

Nucleic acids that can be used in RNAi technology preferably comprise double-stranded RNA molecules that have a sequence that is homologous to a sequence of the gene to be inhibited, i.e. corresponds with respect to the sense strand and the antisense strand to the respective sequence section of the gene to be inhibited. For the purposes of the present invention, the term “double-stranded” covers an intramolecular as well as an intermolecular double-strand formation. Preferably, the homologous region lies in the region of the transcript of the gene to be inhibited, that is, in the region of the mRNA (or cDNA) of the gene to be inhibited. The homologous range preferably lies in the encoding region or in 5′ or 3′ UTR region (UTR=untranslated region) of the mRNA of the gene to be inhibited. The nucleic acids according to the invention are preferably double-stranded or partially paired/hybridized RNA molecules.

According to embodiments of the invention, the nucleic acid as a double strand can have blunt ends or sticky ends. Double-stranded nucleic acids that have on the 3′ end of each strand an overhang of 1 to 6, preferably 1 or 2 nucleotides have proven to be particularly effective. The overhanging nucleotides are preferably 2′-deoxy nucleotides, particularly preferably 2′-deoxy thymidine residues. Through the use of the 2′-deoxy nucleotides, the costs of the RNA synthesis can be reduced and the resistance of the RNA with respect to the nuclease degradation can be increased. The overhanging nucleotides do not necessarily have to be nucleotides homologous to the target sequence. Nucleic acids with short overhangs are preferred, in particular of 2 nucleotides, in which the overhanging nucleotides of the antisense strand of the double-stranded nucleic acid are complementary to the target sequence. Nucleic acids have proven to be particularly effective that are homologous to a section of this type of the gene to be inhibited and in particular to the corresponding double-stranded mRNA/cDNA, the sense strand of which is limited on the 5′ side by two adenosine residues (A) and on the 3′ side by two thymidine residues (T), a guanosine residue (G) and a cytosine residue (C) or a thymidine residue and a cytidine residue (C). The section limited by AA and TT, AA and GC or AA and TC preferably has a length of 19 to 21, in particular 19 nucleotides and accordingly has the general form AA(N₁₉₋₂₁)TT, AA(N₁₉₋₂₁)GC or AA(N₁₉₋₂₁)TC, wherein N stands for any nucleotide. Nucleic acids are also preferred that are complementary to a section of the gene to be inhibited or to the corresponding double-stranded cDNA or mRNA, which has the general form AA(N₁₉) to AA(N₂₁). Hereby nucleic acids that are homologous to the N₁₉₋₂₁ fragment of the cited regions are particularly preferred. The particularly preferred double-stranded nucleic acids thus have a length of 19 to 21 base pairs, wherein the individual strands forming these double-stranded nucleic acids have on the 3′ side preferably respectively two additional 2′-deoxy nucleotides, in particular two 2′-deoxy thymidine residues so that the dsRNA has 19 to 21 base pairs and per strand two overhanging 2′-deoxy nucleotides. If the gene to be inhibited does not contain a region of the form AA(N₁₉₋₂₁), regions of the form NA(N₁₉₋₂₁) or any fragment of the form N₁₉₋₂₁ is sought. N₁₉₋₂₁ fragments that are limited, e.g., by AA and TT, are preferred, but fundamentally according to the invention all double-stranded nucleic acids are suitable that are at least ≧80%, in some embodiments ≧90%, in some embodiments ≧95%, in some other embodiments ≧98%, in some other embodiments ≧99%, in some still other embodiments ≧99.5%, and in some still further embodiments 100% identical to the target sequence.

Nucleic acids that themselves can inhibit the expression of a representative of the Ras gene family by means of RNAi are preferably double-stranded, have at least a proportion of RNA or comprise RNA and can have a length that corresponds to that of mRNA or cDNA of the respective gene to be inhibited or is shorter. Preferably these nucleic acids have a length of 15 to 49 nucleotides, particularly preferably 17 to 30 nucleotides and very particularly preferably 10 to 23 nucleotides. According to the invention, the at least one nucleic acid can be a siRNA molecule (small interfering RNA). This is a double-stranded RNA molecule with a length of 19 to 25 nucleotides, which has a sequence or is composed thereof that is homologous to a region of the mRNA of the gene that is to be inhibited (as already described above). Alternatively, the at least one nucleic acid can also be an shRNA molecule (small hairpin RNA). An shRNA molecule has two sequence regions that are reversely complementary to one another and can form a double strand with one another in an intramolecular manner. These reversely complementary regions are separated from one another by a region that cannot form an intramolecular double strand and with the formation of a double strand of the first two regions remains as a so-called loop. The reversely complementary regions have a sequence that is homologous to a region of the mRNA of the gene that is to be inhibited (as already described above).

According to some embodiments of the invention, the coating or the cavity filling of the implant can contain a nucleic acid that encodes for a nucleic acid that can inhibit the expression of at least one representative of the Ras gene family by means of RNAi. This nucleic acid is preferably a DNA nucleic acid and, in addition to the sequence that encodes for the nucleic acid, which can inhibit the expression of at least one representative of the Ras gene family by means of RNAi, can comprise further constituents and sequences. Preferably, this nucleic acid comprises regulatory elements, which permits an expression of the encoded nucleic acid. To this end, the nucleic acid can have a promoter e.g. a U6 promoter. The nucleic acid can be present in the form of a vector. This can be a plasmid vector or a viral vector such as, e.g. a vector of adenoviral or lentiviral origin, The vector can comprise further functional elements or sequences that serve the expression of the encoded nucleic acid, e.g., promoters enhancers, etc., as well as those that do not directly serve the expression of the encoded nucleic acid, e.g., selection markers, replication points etc.

Preferably, the implant according to the invention has a coating or cavity filling containing at least one nucleic acid that comprises or is composed of a sequence or the reverse complement thereof, wherein the sequence is selected from sequences with the SEQ ID NOs: 4, 5, 6, 7, 8, 9, 10, 11 and/or 12 or from sequences that are ≧80%, in other embodiments ≧90%, in still other embodiments ≧95%, in still other embodiments ≧98%, an in still other embodiments ≧99%, in still other embodiments ≧99.5% identical to SEQ ID NOs: 4, 5, 6, 7, 8, 9, 10, 11 and/or 12.

The coating or the cavity filling preferably has several different nucleic acids, wherein at least two nucleic acids can inhibit the expression of different representatives of the Ras gene family or encode for nucleic acids of this type. Particularly preferably, the at least two different nucleic acids can inhibit the expression of respectively different genes selected from N-Ras with the SEQ ID NO: 1, K-Ras with the SEQ ID NO: 2 and/or H-Ras with the SEQ ID NO: 3. To this end the coating or cavity filling of some example implants according to the invention can have at least two different nucleic acids, which respectively contain a sequence or are composed thereof selected from:
i) N-Ras inhibiting sequences with the SEQ ID NOs: 4, 5, 6 or N-Ras inhibiting sequences that are ≧80%, preferably ≧90%, particularly preferably ≧95%, in other embodiments ≧98%, in other embodiments ≧99%, in other embodiments ≧99.5% identical to SEQ ID NOs: 4, 5 or 6;
ii) K-Ras inhibiting sequences with the SEQ ID NOs: 7, 8, 9 or K-Ras inhibiting sequences that are ≧80%, preferably ≧90%, particularly preferably ≧95%, in other embodiments ≧98%, in other embodiments ≧99%, in other embodiments ≧99.5% identical to SEQ ID NOs: 7, 8 or 9; and/or
iii) H-Ras inhibiting sequences with the SEQ ID NOs: 10, 11, 12 or H-Ras inhibiting sequences that are ≧80%, preferably ≧90%, particularly preferably ≧95%, in other embodiments ≧98%, in other embodiments ≧99%, in other embodiments ≧99.5% identical to SEQ ID NOs: 10, 11 or 12;
   wherein the sequence of the first of the two different nucleic acids is not selected from the same group i), ii) or iii) as the sequence of the second.

Preferably the coating or cavity filling of the implant according to the invention has a nucleic acid that is formed by an siRNA molecule comprising at least one of the sequences with the SEQ ID NOs: 4, 5, 7, 8, 10 or 11 with the corresponding reverse complement thereto, or an shRNA molecule with a sequence of the SEQ ID NOs: 6, 9 or 12.

The sequences with the SEQ ID NOs: 4 and 5 can together form an siRNA molecule which is suitable for inhibiting the expression of the N-Ras gene with the SEQ ID NO: 1 and can as such siRNA molecule form the nucleic acid in the coating or cavity filling of the implant according to the invention. The sequence with the SEQ ID NO: 6 can represent a shRNA molecule for inhibiting the N-Ras gene with the SEQ ID NO: 1 and can as such form the nucleic acid in the coating or cavity filling of the implant according to the invention.

The sequences with the SEQ ID NOs: 7 and 8 can together form a siRNA molecule which is suitable for inhibiting the expression of the K-Ras gene with the SEQ ID NO: 2 and can as such siRNA molecule form the nucleic acid in the coating or cavity filling of the implant according to the invention. The sequence with the SEQ ID NO: 9 can represent a shRNA molecule for inhibiting the K-Ras gene with the SEQ ID NO: 2 and can as such form the nucleic acid in the coating or cavity filling of the implant according to the invention.

The sequences with the SEQ ID NOs: 10 and 11 can together form a siRNA molecule which is suitable for inhibiting the expression of the H-Ras gene with the SEQ ID NO: 3 and can as such siRNA molecule form the nucleic acid in the coating or cavity filling of the implant according to the invention. The sequence with the SEQ ID NO: 12 can represent a shRNA molecule for inhibiting the H-Ras gene with the SEQ ID NO: 3 and can as such form the nucleic acid in the coating or cavity filling of the implant according to the invention.

Methods for producing the above-referenced nucleic acids are known to one skilled in the art.

Embodiments of the present invention relate to nucleic acids containing or composed of a sequence with the SEQ ID NOs: 4, 5, 6, 7, 8, 9, 10, 11 and/or 12 or a sequence that is ≧80%, preferably ≧90%, particularly preferably ≧95%, in other embodiments ≧98%, in other embodiments ≧99%, in other embodiments ≧99.5% identical to SEQ ID NOs: 4, 5, 6, 7, 8, 9, 10, 11 and/or 12 or the reverse complement thereof for preventing and/or inhibiting restenosis.

By way of further illustration, some aspects of the invention are described in more detail below based on exemplary embodiments.

The siRNA or shRNA are to be applied to the example implants of the invention in a manner that ensures a local release and thus a local uptake into the cells.
1. Coating with siRNA/shRNA in matrix, from which the siRNA/shRNA elutes. Wherein the entire range of natural and synthetic as well as degradable and non-degradable polymers can be used here.
2. Coating with siRNA/shRNA in cavities. Wherein the siRNA/shRNA optionally is embedded in a matrix and the cavities are optionally open or covered (top coat or the like). Hydrogels above all can be used here which contain the RNA in quantities between 2-50% (m/m) preferably 10-25%. The substances can leave the cavities through swelling and/or diffusion, wherein the substance can also be released from the gel as nanoparticles (see below).
3. Coating with siRNA mixture in nanoparticles.
4. Release of siRNA mixtures in nanoparticles via suitable catheter systems.

Exemplary embodiments for the insertion of the RNA sequences into nanoparticles are provided below.

Mixture of RNA with polyethylene imine: the positively charged polymer with the negatively charged RNA molecules gives precipitates insoluble in water, which with a corresponding stirring rate (300 RPM) and addition of detergents such as 0.1% Triton X100 precipitate as nanoparticles.

Alternatively, instead of the polyethylene imine, polylysine can be used, which likewise represents a polycation.

Chitosan is a further example of a polycation. 5 mg chitosan to this end is placed in 1 ml of a 2% acetic acid solution. 1 ml RNA solution (5.5 mg/ml) is added to this solution and thereby vigorously vortexed. The fine precipitate is taken up in PBS buffer 50 mM and further processed.

In addition to these hydrophilic polymers, hydrophobic polymers such as PLLA, PLDLA, PLGA can also be used. In this case, e.g., a water/oil/water (W/O/W) emulsion or a (W/O/O) emulsion should be used via a special method in order to convert (capture) the purely water-soluble RNA into a water-insoluble polymer.

### Example of a W/O/W Emulsion:

### First Emulsion

Dissolve PLLA L210 0.1-0.2 g in 100 ml chloroform. Addition of 1-2 ml RNA solution 5 mg/ml. The two-phase mixture is vigorously stirred, Ultra-Turrax 10,000 RPM. The polymer is in the organic phase and the RNA in the aqueous phase.

### Second Emulsion

Only 100 ml water that contains a surfactant Triton X100 0.2% is added to the first emulsion. The RNA is present encapsulated in the polymer, in the aqueous phase virtually nothing is dissolved.

### Processing:

After withdrawal of the solvent, the polymer/RNA nanoparticles can be obtained.

The nanoparticles produced in this manner are directly suspended in a hydrogel matrix or filled into cavities. The nanoparticles, in particular those with a positive charge excess on the surface are suitable for locking the nucleic acid information in the cell core

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implant with one or more of a coating or a cavity filling containing a nucleic acid which
i) inhibits the expression of at least one representative of the Ras gene family by RNA interference; or
ii) encodes for a nucleic acid that inhibits the expression of at least one member of the Ras gene family by RNA interference.

2. An implant according to claim 1, characterized in that the implant is comprised of a biocorrodible metallic material.

3. An implant according to claim 2, characterized in that the biocorrodible metallic material is a magnesium alloy.

4. An implant according to claim 1, characterized in that the nucleic acid inhibits the expression of at least one of the genes N-Ras with the SEQ ID NO: 1, K-Ras with the SEQ ID NO: 2 and H-Ras with the SEQ ID NO: 3 or a gene having cDNA that is ≧80%, identical to one of the sequences with one or more of the SEQ ID NOs: 1, 2 and 3.

5. An implant according to claim 1, characterized in that the nucleic acid inhibits the expression of at least one of the genes N-Ras with the SEQ ID NO: 1, K-Ras with the SEQ ID NO: 2 and H-Ras with the SEQ ID NO: 3 or a gene having cDNA that is ≧95%, identical to one of the sequences with one or more of the SEQ ID NOs: 1, 2 and 3.

6. An implant according to claim 1, characterized in that the nucleic acid comprises a sequence or the reverse complement thereof, wherein the sequence is selected from sequences with one or more of the SEQ ID NOs: 4, 5, 6, 7, 8, 9, 10, 11 and 12 or from sequences that are ≧80% identical to one or more of SEQ ID NOs: 4, 5, 6, 7, 8, 9, 10, 11 and 12.

7. An implant according to claim 1, characterized in that the nucleic acid comprises a sequence or the reverse complement thereof, wherein the sequence is selected from sequences with one or more of the SEQ ID NOs: 4, 5, 6, 7, 8, 9, 10, 11 and 12 or from sequences that are ≧95% identical to one or more of SEQ ID NOs: 4, 5, 6, 7, 8, 9, 10, 11 and 12.

8. An implant according to claim 1, characterized in that the coating or cavity filling of the implant has at least two different nucleic acids, wherein the two different nucleic acids inhibit the expression of different representatives of the Ras gene family.

9. An implant according to claim 1, characterized in that the coating or the cavity filling of the implant has at least two different nucleic acids which respectively contain a sequence selected from:
i) N-Ras inhibiting sequences with the SEQ ID NOs: 4, 5, 6 or N-Ras inhibiting sequences that are ≧80% identical to SEQ ID NOs: 4, 5 or 6;
ii) K-Ras inhibiting sequences with the SEQ ID NOs: 7, 8, 9 or K-Ras inhibiting sequences that are ≧80% identical to SEQ ID NOs: 7, 8 or 9; and/or
iii) H-Ras inhibiting sequences with the SEQ ID NOs: 10, 11, 12 or H-Ras inhibiting sequences that are ≧80% identical to SEQ ID NOs: 10, 11 or 12;
wherein the respective sequence of the two different nucleic acids is not selected from the same group i), ii) or iii).

10. An implant according to claim 1, characterized in that the coating or the cavity filling of the implant has at least two different nucleic acids which respectively contain a sequence selected from:
i) N-Ras inhibiting sequences with the SEQ ID NOs: 4, 5, 6 or N-Ras inhibiting sequences that are ≧95% identical to SEQ ID NOs: 4, 5 or 6;
ii) K-Ras inhibiting sequences with the SEQ ID NOs: 7, 8, 9 or K-Ras inhibiting sequences that are ≧95% identical to SEQ ID NOs: 7, 8 or 9; and/or
iii) H-Ras inhibiting sequences with the SEQ ID NOs: 10, 11, 12 or H-Ras inhibiting sequences that are ≧95% identical to SEQ ID NOs: 10, 11 or 12;
wherein the respective sequence of the two different nucleic acids is not selected from the same group i), ii) or iii).

11. An implant according to claim 1, characterized in that the nucleic acid is an siRNA molecule comprising a double strand formed from one of the sequences SEQ ID NOs: 4, 5, 7, 8, 10 or 11 and the corresponding reverse complement thereto or an shRNA molecule with a sequence with the SEQ ID NOs: 6, 9 or 12.

12. An implant according to claim 1, characterized in that the nucleic acid is a DNA.

13. An implant according to claim 1, characterized in that the nucleic acid comprises a vector.

14. An implant according to claim 1, characterized in that the coating or the cavity filling of the implant contains the nucleic acid in the form of nanoparticles loaded with nucleic acid.

15. An implant according to claim 1, characterized in that the nucleic acid is embedded in an organic matrix which is applied to the implant as a coating or cavity filling.

16. An implant according to claim 1, characterized in that the implant is a stent.

17. A nucleic acid containing a sequence with one or more of the SEQ ID NOs: 4, 5, 6, 7, 8, 9, 10, 11 and 12 or of a sequence that is ≧80% identical to SEQ ID NOs: 4, 5, 6, 7, 8, 9, 10, 11 and/or 12 or the reverse complement thereof for preventing and/or inhibiting restenosis.

18. A nucleic acid as defined by claim 17 wherein the nucleic acid contains a sequence with one or more of the SEQ ID NOs: 4, 5, 6, 7, 8, 9, 10, 11 and 12 or of a sequence that is ≧95% identical to one or more of SEQ ID NOs: 4, 5, 6, 7, 8, 9, 10, 11 and 12 or the reverse complement thereof for preventing and/or inhibiting restenosis.

19. A stent comprising:
a base body made of a biocorrodible magnesium alloy;
at least one nucleic acid that inhibits the expression of at least one representative of the Ras gene family by RNA interference or encodes for a nucleic acid that inhibits the expression of at least one of the Ras gene family by RNA interference; and,
wherein the at least one nucleic acid is provided as one or more of a coating on the base body or as a filling in a base body cavity.

20. A stent as defined by claim 19 wherein the at least one nucleic acid comprises two different nucleic acids which each contain a sequence selected from:
i) N-Ras inhibiting sequences with the SEQ ID NOs: 4, 5, 6 or N-Ras inhibiting sequences that are ≧80% identical to SEQ ID NOs: 4, 5 or 6;
ii) K-Ras inhibiting sequences with the SEQ ID NOs: 7, 8, 9 or K-Ras inhibiting sequences that are ≧80% identical to SEQ ID NOs: 7, 8 or 9;
iii) H-Ras inhibiting sequences with the SEQ ID NOs: 10, 11, 12 or H-Ras inhibiting sequences that are ≧80% identical to SEQ ID NOs: 10, 11 or 12;
wherein the respective sequence of the two different nucleic acids is not selected from the same group i), ii) or iii).
